(19) United States
(12) Patent Application Publication
(10) Pub. No.: US 2023/0141118 A1
(43) Pub. Date: May 11, 2023

(54) METHODS OF USING SOLID DISPERSIONS OF RIFAXIMIN FOR THE TREATMENT OF SICKLE CELL DISEASE

(71) Applicant: Bausch Health Ireland Limited, Dublin (IE)

(72) Inventors: Christopher Allen, Verona, NJ (US); Howard Franklin, Richboro, PA (US); Zeev Heimanson, Calabasas, CA (US); Robert J. Israel, Suffern, NY (US); Thejasvi Ramakrishna, Flemington, NJ (US)

(21) Appl. No.: 17/918,374

(22) PCT Filed: Apr. 13, 2021
(86) PCT No.: PCT/EP2021/059579

§ 371 Date: Oct. 12, 2022

Related U.S. Application Data
(60) Provisional application No. 63/009,043, filed on Apr. 13, 2020.

(51) Int. Cl. A61K 31/496; A61K 9/14; A61K 31/485; A61K 31/17; A61K 31/198

(52) U.S. Cl. CPC A61K 31/496; A61K 9/146; A61K 31/485; A61K 31/17; A61K 31/198

(57) ABSTRACT

Provided herein are solid dispersions comprising rifaximin and methods of using the same for the treatment of sickle cell disease (SCD).

### RELATED APPLICATIONS

This application claims the benefit of U.S. Provisional Application No. 63/009,043, filed Apr. 13, 2020, the entire contents of which are incorporated herein by reference.

### BACKGROUND

Sickle cell disease (SCD) affects approximately 100,000 Americans and while the standard of care has improved, there remains an urgent need for new and efficacious SCD therapies.

### SUMMARY

The invention described herein meets the needs in the field by providing a treatment for sickle cell disease (SCD) through the administration of a soluble solid dispersion (SSD) of rifaximin to patients in need thereof.

Patients having SCD may have recurrent painful vaso-occlusive crises (VOCs), which is the most common clinical manifestation of SCD. VOC occurs when the patient's microcirculation is obstructed by sickled red blood cells (RBCs), which may result in ischemic injury, ulcers, priapism, organ damage, and spontaneous abortion. Furthermore, patients having SCD may, overall, have a poor quality of life and a shortened lifespan.

Neutrophils have been implicated in regulating VOC in SCD patients. SCD patients with WBC>15×10⁹/L are more likely to develop stroke, acute chest syndrome, and premature death. Neutrophils in SCD patients are also shown to exhibit increased levels of activation molecules, including CD64 and CD11b/CD18, with their sera having elevated levels of soluble CD62L. A subset of neutrophils known as circulating aged neutrophils (CANs) are substantially elevated. CANs are characterized by having a high surface expression of CXCR4 and low CD62L. Activated and aged neutrophils may be immobilized in the circulatory system on the endothelium and form the nidus for the adhesion of sickled RBCs, which may lead to VOC.

It has been reported that modulating intestinal microbial composition may be a therapeutic option in treating SCD patients to reduce VOC through the reduction of activated and aged neutrophils. In one study, it was found that a 550 mg dose of rifaximin (i.e., XIFAXAN® 550 mg), delivered BID, was capable of reducing CANs in SCD patients (Clinical Trial Identifier: NCT03719729). Furthermore, when SCD patients received a 550 mg dose of rifaximin (i.e., XIFAXAN® 550 mg) BID for 6 months, the result was a decrease in the number of VOCs, and thus an increased quality of life.

XIFAXAN® is commercially available in the United States as a 200 mg formulation and a 550 mg formulation. The 200 mg formulation is provided for the treatment of travelers' diarrhea (TD) caused by noninvasive strains of E. coli in adult and pediatric patients 12 years of age and older by administration of one 200 mg tablet TID for 3 days. The 550 mg formulation is provided for the reduction of risk of overt hepatic encephalopathy (HE) recurrence in adults and for the treatment of irritable bowel syndrome with diarrhea (IBS-D) in adults. For the treatment of HE, one 550 mg tablet is administered BID. For the treatment of IBS-D, one 550 mg tablet is administered TID for 14 days. In both XIFAXAN® 200 mg and 550 mg, the rifaximin is delivered locally to the GI tract and is not systemically absorbed.

The rifaximin soluble solid dispersions (SSDs) described herein are provided to increase the gastrointestinal luminal solubility of rifaximin, while minimizing systemic exposure. Accordingly, the rifaximin SSDs described herein provide a therapy for treating SCD in a patient by, for example, and without being limited to any one theory of the invention, (1) reducing levels of elevated circulating aged neutrophils (CANs), and/or (2) reducing or preventing the occurrence of vaso-occlusive crises (VOCs). As opposed to prior therapies, and the knowledge in the art, the benefits offered by the invention described herein are provided at a substantially reduced dosage of rifaximin while providing clinical benefit.

In an embodiment, the invention described herein includes a method of treating sickle cell disease (SCD) in a patient in need thereof comprising administering at least one rifaximin SSD composition to the patient. In some embodiments, the at least one rifaximin SSD composition comprises at least one rifaximin SSD tablet. In some embodiments, the method of treating sickle cell disease (SCD) comprises reducing elevated levels of circulating aged neutrophils (CANs) in the patient. In some embodiments, the method of treating sickle cell disease (SCD) in a patient comprises treating vaso-occlusive crisis (VOC) in the patient. In some embodiments, treating vaso-occlusive crisis (VOC) in the patient comprises (1) alleviating one or more symptoms of VOC in the patient; (2) reducing or preventing the occurrence of VOCs in the patient; (3) reducing the duration or severity of VOC in the patient; and/or (4) mediating or otherwise reducing the patient's opioid usage during VOC. In some embodiments, the method of treating sickle cell (SCD) in the patient comprises alleviating one or more symptoms of vaso-occlusive crisis (VOC) in the patient. In some embodiments, the method of treating sickle cell disease (SCD) in the patient comprises reducing or preventing the occurrence of vaso-occlusive crises (VOCs) in the patient. In some embodiments, the method of treating sickle cell disease (SCD) comprises reducing the duration or severity of VOC in the patient. In some embodiments, the method of treating sickle cell disease (SCD) in the patient comprises mediating or otherwise reducing the patient's opioid usage during vaso-occlusive crisis (VOC) in the patient.

In some embodiments, the at least one rifaximin SSD composition comprises an immediate release (IR) tablet and/or a sustained extended release tablet. In some embodiments, the at least one rifaximin SSD composition comprises a 40 mg dosage of rifaximin or an 80 mg dosage of rifaximin. In some embodiments, the at least one rifaximin SSD composition comprises a 40 mg rifaximin SSD IR tablet, an 80 mg rifaximin SSD IR tablet, a 40 mg SSD SER tablet, or an 80 mg SSD SER tablet. In some embodiments, the at least one rifaximin SSD composition is a 40 mg rifaximin SSD IR tablet or two or more 40 rifaximin SSD IR tablets.

In some embodiments, the at least one rifaximin SSD composition is delivered QD, BID, TID, or QID to the patient. In some embodiments, the at least one tablet rifaximin SSD composition is delivered QD, BID, TID, or QID to the patient for a period of at least 1, 2, 3, 4, 5, 6, 7, 8, 9, 10, 11, or 12 months. In some embodiments, the at least one rifaximin SSD composition is delivered QD, BID, TID, or QID to the patient for a period of about 1, 2, 3, 4, 5, 6, 7, 8, 9, 10, 11, or 12 months. In some embodiments, the at least one rifaximin SSD composition is delivered QD, BID, TID, or QID to the patient for a period of at most 1, 2, 3, 4, 5, 6, 7, 8, 9, 10, 11, or 12 months. In certain embodiments, the at least one rifaximin SSD composition is delivered QD or BID for at least 6 months. In certain embodiments, the at least one rifaximin SSD composition is delivered QD for at least 6 months. In certain embodiments, the at least one rifaximin SSD composition is delivered BID for at least 6 months.

In some embodiments, the methods described herein further include administering an additional therapeutic agent, such as an SCD therapeutic agent. In some embodiments, the additional therapeutic agent is an SCD therapeutic agent. In some embodiments, the SCD therapeutic agent is selected from the group consisting of hydroxyurea, L-glutamine, hydroxycarbamide, an erythropoietin stimulating agent, an opioid analgesic, and combinations thereof. In some embodiments, the opioid analgesic is selected from the group consisting of morphine, codeine, hydrocodone, hydromorphone, methadone, tramadol, oxycodone, tapentadol, fentanyl, and combinations thereof. In some embodiments, the SCD therapeutic agent comprises an opioid analgesic.

### DETAILED DESCRIPTION

Provided herein are soluble solid dispersions (SSDs) comprising rifaximin and a polymer, such as hydroxypropyl methylcellulose acetate succinate (HPMC-AS), and methods of using the same for the treatment of Sickle Cell Disease (SCD).

### Definitions

The term “solid dispersion” or as used herein refers to a dispersion of rifaximin and an inert carrier matrix in a solid form, i.e., rifaximin is homologously mixed with an inert carrier. The inert matrix is generally hydrophilic (e.g., a polymer such as HPMC-AS) and may be crystalline or amorphous. It will be understood that it is not necessarily the preparation method that governs the properties of the solid dispersion, but rather the molecular arrangement of the contents of the dispersion. Thus, absent an expression to do so, or an incorporation of process restrictions, solid dispersions are not to be limited by the process to which they are made. The terms “solid dispersion”, “soluble solid dispersion”, and the abbreviations “SD” or “SSD” are used interchangeably and each refer to the disclosed solid dispersion of rifaximin.

As used herein the terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The language “therapeutically effective amount” refers to an amount of a composition comprising a solid dispersion of rifaximin effective, upon single or multiple dose administration to the subject to provide a therapeutic benefit to the subject.

As used herein the term “treatment of sickle cell disease (SCD)” may refer to the amelioration, prevention, or reduction in frequency of one or more symptoms of SCD. For example, “treatment of sickle cell disease (SCD)” may refer to the reduction of elevated levels of circulating aged neutrophils (CANs) in a patient, where such levels of CANs are elevated as compared, for example, to levels of CANs that would be expected in a patient who is not diagnosed as having SCD. As another example, the “treatment of sickle cell disease (SCD)” may refer to treating vaso-occlusive crisis (VOC) in the patient, where “treating vaso-occlusive crisis (VOC) or “treating vaso-occlusive crises (VOCs),” as the case may be, may refer to (1) alleviating one or more symptoms of VOC in the patient; (2) reducing or preventing the occurrence of VOCs in the patient; (3) reducing the duration or severity of VOC in the patient; and/or (4) mediating or otherwise reducing the patient's opioid usage during VOC. For example, symptoms of VOC include, without limitation, pain, swelling, ischemic injury, ulcers, priapism, organ damage, and spontaneous abortion. The “treatment of sickle cell disease (SCD)” may refer to preventing the occurrence of vaso-occlusive crisis (VOC) in a patient, such as a patient having a history of VOC (e.g., at least one VOC in the 12 months prior to treatment). The “treatment of sickle cell disease (SCD)” may refer to reducing the occurrence or frequency of vaso-occlusive crisis (VOC) in a patient, such as a patient having a history of VOC. The “treatment of sickle cell disease (SCD)” may refer to reducing the severity of vaso-occlusive crisis (VOC) occurrences in a patient, such as a patient having a history of VOC. The “treatment of sickle cell disease (SCD)” may refer to mediating or reducing a patient's opioid usage during VOC. The mediation or reduction of a patient's opioid usage during VOC may refer to, for example, mediating or reducing the patient's reliance on opioids (e.g., opioid analgesics) for pain management during VOC as compared to the patient's history of reliance on such opioids during VOC. Alternatively, the mediation or reduction of a patient's opioid usage during VOC may refer to mediating or reducing the patient's reliance on opioids (e.g., opioid analgesics) for pain management during VOC as compared to a patient during VOC who is not receiving a rifaximin SSD formulation as described herein.

In the present disclosure, when a numerical value is modified by the term “about”, the exact numerical value is also deemed to be disclosed.

### Compositions

In an embodiment, the present disclosure provides a solid dispersion, such as a rifaximin SSD, comprising rifaximin and a polymer, such as HPMC-AS.

In a second embodiment, the present disclosure provides a solid dispersion comprising rifaximin and HPMC-AS, wherein the HPMC-AS is present in an amount of from about 10 wt % to about 60 wt %, from about 10 wt % to about 50 wt %, from about 10 wt % to about 40 wt %, from about 12 wt % to about 38 wt %, from about 15 wt % to about 35 wt %, from about 16 wt % to about 34 wt %, from about 30 wt % to about 40 wt %, from about 30 wt % to about 35 wt %, from about 33 wt % to about 35 wt %, about 32 wt %, about 33 wt %, about 34 wt %, about 35 wt %, from about 10 wt % to about 20 wt %, from about 13 wt % to about 18 wt %, from about 16 wt % to about 18 wt %, about 15 wt %, about 16 wt %, about 17 wt %, about 18 wt %, from about 40 wt % to about 50 wt %, from about 46 wt % to about 49 wt %, about 46 wt %, about 47 wt %, or about 48 wt %. In one alternative, the amount of HPMC-AS present in the solid dispersion is about 46 wt % to about 49 wt %, about 46 wt %, about 47 wt %, about 48 wt %, from about 33 wt % to about 35 wt %, about 33 wt %, about 34 wt %, about 35 wt %, from about 16 wt % to about 34 wt %, from about 16 wt % to about 18 wt %, about 16 wt %, about 17 wt %, or about 18 wt %. In another alternative, the amount of HPMC-AS present in the solid dispersion is about 46 wt %, about 47 wt %, about 48 wt %, about 33 wt %, about 34 wt %, about 35 wt %, about 16 wt %, about 17 wt %, or about 18 wt % HPMC-AS. In yet another alternative, the amount of HPMC-AS present in the solid dispersion is about 46 wt %, about 47 wt %, or about 48 wt %.

In a third embodiment, the solid dispersion comprises equal amounts of rifaximin and polymer. Thus, for example, the solid dispersion comprises from about 10 wt % to about 60 wt %, from about 10 wt % to about 50 wt %, from about 10 wt % to about 40 wt %, from about 12 wt % to about 38 wt %, from about 15 wt % to about 35 wt %, from about 16 wt % to about 34 wt %, from about 30 wt % to about 40 wt %, from about 30 wt % to about 35 wt %, from about 33 wt % to about 35 wt %, about 32 wt %, about 33 wt %, about 34 wt %, about 35 wt %, from about 10 wt % to about 20 wt %, from about 13 wt % to about 18 wt %, from about 16 wt % to about 18 wt %, about 15 wt %, about 16 wt %, about 17 wt %, about 18 wt %, from about 40 wt % to about 50 wt %, from about 46 wt % to about 49 wt %, about 46 wt %, about 47 wt %, or about 48 wt % rifaximin and HPMC-AS. In another aspect, the solid dispersion comprises from about 46 wt % to about 49 wt %, about 46 wt %, about 47 wt %, about 48 wt %, from about 33 wt % to about 35 wt %, about 33 wt %, about 34 wt %, about 35 wt %, from about 16 wt % to about 34 wt %, from about 16 wt % to about 18 wt %, about 16 wt %, about 17 wt %, or about 18 wt % rifaximin and HMPC-AS. In another aspect, the solid dispersion comprises about 46 wt %, about 47 wt %, about 48 wt %, about 33 wt %, about 34 wt %, about 35 wt %, about 16 wt %, about 17 wt %, or about 18 wt % rifaximin and HPMC-AS. In yet another aspect, the solid dispersion comprises about 46 wt %, about 47 wt %, or about 48 wt % rifaximin and HPMC-AS.

In a fourth embodiment, the solid dispersion comprising rifaximin and HPMC-AS further comprises poloxamer 407 (e.g., Pluronic® F-127), wherein the remaining components and amounts present in the solid dispersion are as described in the second or third embodiment.

In a fifth embodiment, the solid dispersion comprising rifaximin and HPMC-AS further comprises poloxamer 407 (e.g., Pluronic® F-127) in an amount from about 0.5 wt % to about 7 wt %, from about 0.5 wt % to about 5 wt %, from about 1 wt % to about 5 wt %, from about 1 wt % to about 4 wt %, from about 2 wt % to about 4 wt %, from about 4 wt % to about 6 wt %, from about 3 wt % to about 5 wt %, from about 2 wt % to about 4 wt %, from about 1 wt % to about 2 wt %, about 1 wt %, about 2 wt %, about 3 wt %, about 4 wt %, about 5 wt %, about 5.5 wt %, or about 6 wt %, wherein the remaining components and amounts present in the solid dispersion are as described in the second, third, or fourth embodiment. In one alternative, the solid dispersion comprises about 1 wt %, about 2 wt %, about 3 wt %, about 4 wt %, about 5 wt %, about 5.5 wt %, about 5.6 wt %, about 5.7 wt %, or about 6 wt % poloxamer 407 (e.g., Pluronic® F-127), wherein the remaining components and amounts present in the solid dispersion are as described in the second, third, or fourth embodiment. In yet another alternative, the solid dispersion of rifaximin comprises about 5 wt %, about 5.5 wt %, or about 6 wt % poloxamer 407 (e.g., Pluronic® F-127), wherein the remaining components and amounts present in the solid dispersion are as described in the second, third, or fourth embodiment.

In a sixth embodiment, provided are pharmaceutical compositions, such as rifaximin SSD compositions, comprising the solid dispersion of any one of the first, second, third, fourth, or fifth embodiment.

In a seventh embodiment, provided are pharmaceutical compositions comprising the solid dispersions of any one of the first, second, third, fourth, or fifth embodiments together with croscarmellose sodium (crosslinked carboxymethyl cellulose sodium).

In an eighth embodiment, provided are pharmaceutical compositions comprising the solid dispersions of any one of the first, second, third, fourth, or fifth embodiments together with croscarmellose sodium in an amount from about 2 wt % to about 15 wt %, from about 3 wt % to about 14 wt %, from about 4 wt % to about 14 wt %, from about 2 wt % to about 13 wt %, from about 3 wt % to about 13 wt %, from about 4 wt % to about 13 wt %, from about 11 wt % to about 14 wt %, from about 12 wt % to about 14 wt %, from about 4 wt % to about 10 wt %, about 12 wt %, about 12.5 wt %, about 13 wt %, about 13.5 wt %, from about 4 wt % to about 6 wt %, about 5 wt %, from about 8% to about 10 wt %, or about 9 wt % based on the total amount (wt %) of components in the pharmaceutical composition, wherein the remaining components and amounts present in the pharmaceutical composition include and are as described in the second, third, fourth, fifth, sixth, or seventh embodiment. In one alternative, the croscarmellose sodium is present in an amount from about 4 wt % to about 14 wt %, from about 12 wt % to about 14 wt %, about 13 wt %, from about 4 wt % to about 6 wt %, about 5 wt %, from about 8% to about 10 wt %, or about 9 wt % based on the total amount (wt %) of components in the pharmaceutical composition, wherein the remaining components and amounts present in the pharmaceutical composition include and are as described in the second, third, fourth, fifth, sixth, or seventh embodiment. In another alternative, the croscarmellose sodium is present in an amount from of rifaximin is about 13 wt %, about 5 wt %, or about 9 wt % based on the total amount (wt %) of components in the pharmaceutical composition, wherein the remaining components and amounts present in the pharmaceutical composition include and are as described in the second, third, fourth, fifth, sixth, or seventh embodiment.

In a ninth embodiment, the pharmaceutical compositions described herein further comprise microcrystalline cellulose, wherein the remaining components and amounts present in the pharmaceutical composition include and are as described in the second, third, fourth, fifth, sixth, seventh, or eighth embodiment.

In a tenth embodiment, the pharmaceutical compositions described herein further comprise microcrystalline cellulose present in an amount from about 5 wt % to about 60 wt %, from about 10 wt % to about 55 wt %, from about 5 wt % to about 15 wt %, from about 8 wt % to about 13 wt %, from about 10 wt % to about 12 wt %, from about 10 wt % to about 19 wt %, about 11 wt %, from about 15 wt % to about 25 wt %, from about 17 wt % to about 19 wt %, about 18 wt %, from about 40 wt % to about 60 wt %, from about 45 wt % to about 55 wt %, from about 49 wt % to about 55 wt %, from about 49 wt % to about 51 wt %, from about 53 wt % to about 55 wt %, about 50 wt %, or about 54 wt % based on the total amount (wt %) of components in the pharmaceutical composition, wherein the remaining components and amounts present in the pharmaceutical composition include and are as described in the second, third, fourth, fifth, sixth, seventh, eighth, or ninth embodiment.

In an eleventh embodiment, the pharmaceutical compositions described herein further comprise colloidal silicon dioxide, wherein the remaining components and amounts present in the pharmaceutical composition include and are as described in the second, third, fourth, fifth, sixth, seventh, eighth, ninth, or tenth embodiment.

In a twelfth embodiment, the pharmaceutical compositions described herein further comprise colloidal silicon dioxide present in an amount from about 0.1 wt % to about 0.3 wt %, from about 0.15 wt % to about 0.25 wt %, or about 0.2 wt % based on the total amount (wt %) of components in the pharmaceutical composition, wherein the remaining components and amounts present in the pharmaceutical composition include and are as described in the second, third, fourth, fifth, sixth, seventh, eighth, ninth, tenth, or eleventh embodiment.

In a thirteenth embodiment, the pharmaceutical compositions described herein further comprise magnesium stearate, wherein the remaining components and amounts present in the pharmaceutical composition include and are as described in the second, third, fourth, fifth, sixth, seventh, eighth, ninth, tenth, eleventh, or twelfth embodiment.

In a fourteenth embodiment, the pharmaceutical compositions described herein further comprise magnesium stearate present in an amount from about 0.3 wt % to about 0.6 wt %, from about 0.4 wt % to about 0.6 wt %, from about 0.45 wt % to about 0.55 wt %, about 0.45 wt %, about 0.47 wt %, or about 0.49 wt % based on the total amount (wt %) of components in the pharmaceutical composition, wherein the remaining components and amounts present in the pharmaceutical composition include and are as described in the second, third, fourth, fifth, sixth, seventh, eighth, ninth, tenth, eleventh, twelfth, or thirteenth embodiment.

In a fifteenth embodiment, provided is a pharmaceutical composition comprising from about 33 wt % to about 35 wt % rifaximin; from about 33 wt % to about 35 wt % HPMC-AS; from about 3 wt % to about 5 wt % poloxamer 407; from about 4 wt % to about 14 wt % croscarmellose sodium; from about 10 wt % to about 19 wt % microcrystalline cellulose; from about 0.15 wt % to about 0.25 wt % colloidal silicon dioxide; and from about 0.45 wt % to about 0.55 wt % magnesium stearate.

In a sixteenth embodiment, provided is a pharmaceutical composition according to the fifteenth embodiment, wherein comprising the croscarmellose sodium is present in an amount of from about 12 wt % to about 14 wt %. Alternatively, provided is a pharmaceutical composition according to the fifteenth embodiment, wherein comprising the croscarmellose sodium is present in an amount of about 13%.

In a seventeenth embodiment, provided is a pharmaceutical composition according to the fifteenth or sixteenth embodiment wherein the microcrystalline cellulose is present in an amount from about 10 wt % to about 12 wt %. Alternatively, provided is a pharmaceutical composition according to the fifteenth or sixteenth embodiment wherein the microcrystalline cellulose is present in an amount of about 11 wt %.

In an eighteenth embodiment, provided is a pharmaceutical composition according to the fifteenth embodiment, wherein the croscarmellose sodium is present in an amount from about 4 wt % to about 6 wt %. Alternatively, provided is a pharmaceutical composition according to the fifteenth embodiment, wherein the croscarmellose sodium is present in an amount of about 5 wt %.

In a nineteenth embodiment, provided is a pharmaceutical composition according to the fifteenth or eighteenth embodiment, wherein the microcrystalline cellulose is present in an amount from about 17 wt % to about 19 wt %. Alternatively, provided is a pharmaceutical composition according to the fifteenth or eighteenth embodiment, wherein the microcrystalline cellulose is present in an amount of about 18 wt %.

In a twentieth embodiment, provided is a pharmaceutical composition according to the fifteenth, sixteenth, seventeenth, eighteenth, or nineteenth embodiment, wherein the poloxamer 407 is present in an amount of about 4%.

In a twenty-first embodiment, provided is a pharmaceutical composition according to the fifteenth, sixteenth, seventeenth, eighteenth, nineteenth, or twentieth embodiment, wherein the colloidal silicon dioxide is present in an amount of about 0.20 wt %.

In a twenty-second embodiment, provided is a pharmaceutical composition according to the fifteenth, sixteenth, seventeenth, eighteenth, nineteenth, twentieth, or twenty-first embodiment, wherein the magnesium stearate is present in an amount of about 0.50 wt %.

In a twenty-third embodiment, provided is a pharmaceutical composition according to the fifteenth, sixteenth, seventeenth, eighteenth, nineteenth, twentieth, twenty-first, or twenty-second embodiment, wherein the rifaximin is present in an amount of about 34%.

In a twenty-fourth embodiment, provided is a pharmaceutical composition according to the fifteenth, sixteenth, seventeenth, eighteenth, nineteenth, twentieth, twenty-first, twenty-second, or twenty-third embodiment, wherein the HPMC-AS is present in an amount of about 34%.

In a twenty-fifth embodiment, provided is a pharmaceutical composition according to the fifteenth, sixteenth, seventeenth, eighteenth, nineteenth, twentieth, twenty-first, twenty-second, twenty-third embodiment, or twenty fourth embodiment, wherein the total amount of rifaximin is about 80 mg.

In a twenty-sixth embodiment, provided is a pharmaceutical composition comprising from about 16 wt % to about 18 wt % rifaximin; from about 16 wt % to about 18 wt % HPMC-AS; from about 1 wt % to about 2 wt % poloxamer 407; from about 4 wt % to about 10 wt % croscarmellose sodium; from about 49 wt % to about 55 wt % microcrystalline cellulose; from about 0.15 wt % to about 0.25 wt % colloidal silicon dioxide; and from about 0.45 wt % to about 0.55 wt % magnesium stearate.

In a twenty-seventh embodiment, provided is a pharmaceutical composition according to the twenty-sixth embodiment, wherein the croscarmellose sodium is present in an amount from about 8 wt % to about 10 wt %. Alternatively, provided is a pharmaceutical composition according to the twenty-sixth embodiment, wherein the croscarmellose sodium is present in an amount of about 9 wt %.

In a twenty-eighth embodiment, provided is a pharmaceutical composition according to the twenty-sixth or twenty-seventh embodiment, wherein the microcrystalline cellulose is present in an amount from about 49 wt % to about 51 wt %. Alternatively, provided is a pharmaceutical composition according to the twenty-sixth or twenty-seventh embodiment, wherein the microcrystalline cellulose is present in an amount of about 51 wt %.

In a twenty-ninth embodiment, provided is a pharmaceutical composition according to the twenty-sixth embodiment, wherein the croscarmellose sodium is present in an amount from about 4 wt % to about 6 wt %. Alternatively, provided is a pharmaceutical composition according to the twenty-sixth embodiment, wherein the croscarmellose sodium is present in an amount of about 5 wt %.

In a thirtieth embodiment, provided is a pharmaceutical composition according to the twenty-sixth or twenty-ninth embodiment, wherein the microcrystalline cellulose is present in an amount from about 53 wt % to about 55 wt %. Alternatively, provided is a pharmaceutical composition according to the twenty-sixth or twenty-ninth embodiment, wherein the microcrystalline cellulose is present in an amount of about 54 wt %.

In a thirty-first embodiment, provided is a pharmaceutical composition according to the twenty-sixth, twenty-seventh, twenty-eighth, twenty-ninth, or thirtieth embodiment, wherein colloidal silicon dioxide is present in an amount of about 0.20 wt %.

In a thirty-second embodiment, provided is a pharmaceutical composition according to the twenty-sixth, twenty-seventh, twenty-eighth, twenty-ninth, thirtieth, or thirty-first embodiment, wherein the magnesium stearate is present in an amount of about 0.50 wt %.

In a thirty-third embodiment, provided is a pharmaceutical composition according to the twenty-sixth, twenty-seventh, twenty-eighth, twenty-ninth, thirtieth, thirty-first, or thirty-second embodiment, wherein the rifaximin is present in an amount of about 17 wt %.

In a thirty-fourth embodiment, provided is a pharmaceutical composition according to the twenty-sixth, twenty-seventh, twenty-eighth, twenty-ninth, thirtieth, thirty-first, thirty-second, or thirty-third embodiment, wherein the HMPC-AS is present in an amount of about 17 wt %.

In a thirty-fifth embodiment, provided is a pharmaceutical composition according to the twenty-sixth, twenty-seventh, twenty-eighth, twenty-ninth, thirtieth, thirty-first, thirty-second, thirty-third, or thirty-fourth embodiment, wherein the total amount of rifaximin is 40 mg.

In a thirty-sixth embodiment, the pharmaceutical compositions described herein are in the form of a tablet.

In a thirty-seventh embodiment, the pharmaceutical compositions described herein are in the form of a tablet, and are immediate release or sustained extended release. In one alternative, the pharmaceutical composition is an immediate release tablet.

In a thirty-eighth embodiment, the pharmaceutical compositions described herein are film coated. Coatings are known to those of skill in the art and may immediate release or sustained release coatings. An example of a film coating is Opadry II Blue 85F90614 by Colorcon®.

Other solid dispersions and pharmaceutical compositions included in the present disclosure are described in the Exemplification section below.

### Uses, Formulation and Administration Dosage Forms

According to other embodiments, the present disclosure relates to a method of using the disclosed solid dispersions and pharmaceutical compositions thereof (e.g., rifaximin SSD compositions) for the treatment of sickle cell disease (SCD) in patients in need thereof.

Suitable dosage forms that can be used with the solid dispersions and compositions herein include, but are not limited to, capsules, tablets, mini-tablets, beads, beadlets, pellets, granules, granulates, and powder. Suitable dosage forms may be coated, for example using an enteric coating. In some embodiments, the solid dispersions and compositions are formulated as tablets, caplets, or capsules. In one embodiment, the solid dispersions and compositions are formulated as a tablet.

Provided compositions may be formulated such that a dosage of between 0.001-100 mg/kg body weight/day of the inhibitor can be administered to a patient receiving these compositions. It should also be understood that a specific dosage and treatment regimen for any particular patient will depend upon a variety of factors, including age, body weight, general health, sex, diet, time of administration, rate of excretion, drug combination, the judgment of the treating physician, and the severity of the particular disease being treated.

In an embodiment, the invention described herein includes a method of treating sickle cell disease (SCD) in a patient in need thereof comprising administering at least one rifaximin SSD composition to the patient. In some embodiments, the method of treating sickle cell disease (SCD) comprises reducing elevated levels of circulating aged neutrophils (CANs) in the patient. In some embodiments, the method of treating sickle cell disease (SCD) in a patient comprises treating vaso-occlusive crisis (VOC) in the patient. In the methods described herein, the at least one rifaximin SSD composition may be at least one rifaximin SSD tablet.

In some embodiments, treating vaso-occlusive crisis (VOC) in the patient comprises (1) alleviating one or more symptoms of VOC in the patient; (2) reducing or preventing the occurrence of VOCs in the patient; (3) reducing the duration or severity of VOC in the patient; and/or (4) mediating or otherwise reducing the patient's opioid usage during VOC. In some embodiments, the method of treating sickle cell (SCD) in the patient comprises alleviating one or more symptoms of vaso-occlusive crisis (VOC) in the patient. In some embodiments, the method of treating sickle cell disease (SCD) in the patient comprises reducing or preventing the occurrence of vaso-occlusive crises (VOCs) in the patient. In some embodiments, the method of treating sickle cell disease (SCD) in the patient comprises reducing the duration or severity of VOC in the patient. In some embodiments, the method of treating sickle cell disease (SCD) in the patient comprises mediating or otherwise reducing the patient's opioid usage during vaso-occlusive crisis (VOC) in the patient.

In some embodiments, the at least one rifaximin SSD composition comprises at least one rifaximin SSD tablet. In some embodiments, the at least one rifaximin SSD composition comprises an immediate release (IR) tablet and/or a sustained extended release tablet. In some embodiments, the at least one rifaximin SSD composition comprises a 40 mg dosage of rifaximin or an 80 mg dosage of rifaximin. In some embodiments, the at least one rifaximin SSD composition comprises a 40 mg rifaximin SSD IR tablet, an 80 mg rifaximin SSD IR tablet, a 40 mg SSD SER tablet, or an 80 mg SSD SER tablet. In some embodiments, the at least one rifaximin SSD composition is a 40 mg rifaximin SSD IR tablet.

In some embodiments, the at least one rifaximin SSD composition is delivered QD, BID, TID, or QID to the patient. In some embodiments, the at least one tablet rifaximin SSD composition is delivered QD, BID, TID, or QID to the patient for a period of at least 1, 2, 3, 4, 5, 6, 7, 8, 9, 10, 11, or 12 months. In some embodiments, the at least one rifaximin SSD composition is delivered QD, BID, TID, or QID to the patient for a period of about 1, 2, 3, 4, 5, 6, 7, 8, 9, 10, 11, or 12 months. In some embodiments, the at least one rifaximin SSD composition is delivered QD, BID, TID, or QID to the patient for a period of at most 1, 2, 3, 4, 5, 6, 7, 8, 9, 10, 11, or 12 months. In certain embodiments, the at least one rifaximin SSD composition is delivered QD or BID for at least 6 months. In certain embodiments, the at least one rifaximin SSD composition is delivered BID for at least 6 months.

In some embodiments, the at least one rifaximin SSD composition comprises a 40 mg rifaximin SSD IR tablet that is delivered BID to the patient for a period of at least 1, 2, 3, 4, 5, 6, 7, 8, 9, 10, 11, or 12 months. In some embodiments, the at least one rifaximin SSD composition comprises a 40 mg rifaximin SSD IR tablet that is delivered BID to the patient for a period of about 1, 2, 3, 4, 5, 6, 7, 8, 9, 10, 11, or 12 months. In some embodiments, the at least one rifaximin SSD composition comprises a 40 mg rifaximin SSD IR tablet that is delivered BID to the patient for a period of at most 1, 2, 3, 4, 5, 6, 7, 8, 9, 10, 11, or 12 months.

In some embodiments, the at least one rifaximin SSD composition comprises two 40 mg rifaximin SSD IR tablets that are delivered BID to the patient for a period of at least 1, 2, 3, 4, 5, 6, 7, 8, 9, 10, 11, or 12 months. In some embodiments, the at least one rifaximin SSD composition comprises two 40 mg rifaximin SSD IR tablets that are delivered BID to the patient for a period of about 1, 2, 3, 4, 5, 6, 7, 8, 9, 10, 11, or 12 months. In some embodiments, the at least one rifaximin SSD composition comprises two 40 mg rifaximin SSD IR tablets that are delivered BID to the patient for a period of at most 1, 2, 3, 4, 5, 6, 7, 8, 9, 10, 11, or 12 months.

In some embodiments, the at least one rifaximin SSD composition comprises an 80 mg rifaximin SSD IR tablet that is delivered BID to the patient for a period of at least 1, 2, 3, 4, 5, 6, 7, 8, 9, 10, 11, or 12 months. In some embodiments, the at least one rifaximin SSD composition comprises an 80 mg rifaximin SSD IR tablet that is delivered BID to the patient for a period of about 1, 2, 3, 4, 5, 6, 7, 8, 9, 10, 11, or 12 months. In some embodiments, the at least one rifaximin SSD composition comprises an 80 mg rifaximin SSD IR tablet that is delivered BID to the patient for a period of at most 1, 2, 3, 4, 5, 6, 7, 8, 9, 10, 11, or 12 months.

In some embodiments, the at least one rifaximin SSD composition comprises a 40 mg rifaximin SSD IR tablet that is delivered QD to the patient for a period of at least 1, 2, 3, 4, 5, 6, 7, 8, 9, 10, 11, or 12 months. In some embodiments, the at least one rifaximin SSD composition comprises a 40 mg rifaximin SSD IR tablet that is delivered QD to the patient for a period of about 1, 2, 3, 4, 5, 6, 7, 8, 9, 10, 11, or 12 months. In some embodiments, the at least one rifaximin SSD composition comprises a 40 mg rifaximin SSD IR tablet that is delivered QD to the patient for a period of at most 1, 2, 3, 4, 5, 6, 7, 8, 9, 10, 11, or 12 months.

In some embodiments, the at least one rifaximin SSD composition comprises two 40 mg rifaximin SSD IR tablets that are delivered QD to the patient for a period of at least 1, 2, 3, 4, 5, 6, 7, 8, 9, 10, 11, or 12 months. In some embodiments, the at least one rifaximin SSD composition comprises two 40 mg rifaximin SSD IR tablets that are delivered QD to the patient for a period of about 1, 2, 3, 4, 5, 6, 7, 8, 9, 10, 11, or 12 months. In some embodiments, the at least one rifaximin SSD composition comprises two 40 mg rifaximin SSD IR tablets that are delivered QD to the patient for a period of at most 1, 2, 3, 4, 5, 6, 7, 8, 9, 10, 11, or 12 months.

In some embodiments, the at least one rifaximin SSD composition comprises an 80 mg rifaximin SSD IR tablet that is delivered QD to the patient for a period of at least 1, 2, 3, 4, 5, 6, 7, 8, 9, 10, 11, or 12 months. In some embodiments, the at least one rifaximin SSD composition comprises an 80 mg rifaximin SSD IR tablet that is delivered QD to the patient for a period of about 1, 2, 3, 4, 5, 6, 7, 8, 9, 10, 11, or 12 months. In some embodiments, the at least one rifaximin SSD composition comprises an 80 mg rifaximin SSD IR tablet that is delivered QD to the patient for a period of at most 1, 2, 3, 4, 5, 6, 7, 8, 9, 10, 11, or 12 months.

In some embodiments, the at least one rifaximin SSD composition comprises a 40 mg rifaximin SSD IR tablet that is delivered TID to the patient for a period of at least 1, 2, 3, 4, 5, 6, 7, 8, 9, 10, 11, or 12 months. In some embodiments, the at least one rifaximin SSD composition comprises a 40 mg rifaximin SSD IR tablet that is delivered TID to the patient for a period of about 1, 2, 3, 4, 5, 6, 7, 8, 9, 10, 11, or 12 months. In some embodiments, the at least one rifaximin SSD composition comprises a 40 mg rifaximin SSD IR tablet that is delivered TID to the patient for a period of at most 1, 2, 3, 4, 5, 6, 7, 8, 9, 10, 11, or 12 months.

In some embodiments, the at least one rifaximin SSD composition comprises two 40 mg rifaximin SSD IR tablets that are delivered TID to the patient for a period of at least 1, 2, 3, 4, 5, 6, 7, 8, 9, 10, 11, or 12 months. In some embodiments, the at least one rifaximin SSD composition comprises two 40 mg rifaximin SSD IR tablets that are delivered TID to the patient for a period of about 1, 2, 3, 4, 5, 6, 7, 8, 9, 10, 11, or 12 months. In some embodiments, the at least one rifaximin SSD composition comprises two 40 mg rifaximin SSD IR tablets that are delivered TID to the patient for a period of at most 1, 2, 3, 4, 5, 6, 7, 8, 9, 10, 11, or 12 months.

In some embodiments, the at least one rifaximin SSD composition comprises an 80 mg rifaximin SSD IR tablet that is delivered TID to the patient for a period of at least 1, 2, 3, 4, 5, 6, 7, 8, 9, 10, 11, or 12 months. In some embodiments, the at least one rifaximin SSD composition comprises an 80 mg rifaximin SSD IR tablet that is delivered TID to the patient for a period of about 1, 2, 3, 4, 5, 6, 7, 8, 9, 10, 11, or 12 months. In some embodiments, the at least one rifaximin SSD composition comprises an 80 mg rifaximin SSD IR tablet that is delivered TID to the patient for a period of at most 1, 2, 3, 4, 5, 6, 7, 8, 9, 10, 11, or 12 months.

In an embodiment, the invention includes a method of treating sickle cell disease (SCD) in a patient in need thereof comprising administering at least one rifaximin SSD composition to the patient BID for a period of at least 1, 2, 3, 4, 5, 6, 7, 8, 9, 10, 11, or 12 months, wherein the at least one rifaximin SSD composition comprises a 40 mg rifaximin SSD IR tablet or an 80 mg rifaximin SSD IR tablet.

In an embodiment, the invention includes a method of treating sickle cell disease (SCD) in a patient in need thereof comprising administering at least one rifaximin SSD composition to the patient BID fora period of about 1, 2, 3, 4, 5, 6, 7, 8, 9, 10, 11, or 12 months, wherein the at least one rifaximin SSD composition comprises a 40 mg rifaximin SSD IR tablet or an 80 mg rifaximin SSD IR tablet.

In an embodiment, the invention includes a method of treating sickle cell disease (SCD) in a patient in need thereof comprising administering at least one rifaximin SSD composition to the patient BID for a period of at most 1, 2, 3, 4, 5, 6, 7, 8, 9, 10, 11, or 12 months, wherein the at least one rifaximin SSD composition comprises a 40 mg rifaximin SSD IR tablet or an 80 mg rifaximin SSD IR tablet.

In an embodiment, the invention includes a method of treating sickle cell disease (SCD) in a patient in need thereof comprising administering at least one rifaximin SSD composition to the patient BID for a period of at least 1, 2, 3, 4, 5, 6, 7, 8, 9, 10, 11, or 12 months, wherein the at least one rifaximin SSD composition comprises at least two 40 mg rifaximin SSD IR tablets.

In an embodiment, the invention includes a method of treating sickle cell disease (SCD) in a patient in need thereof comprising administering at least one rifaximin SSD composition to the patient BID for a period of about 1, 2, 3, 4, 5, 6, 7, 8, 9, 10, 11, or 12 months, wherein the at least one rifaximin SSD composition comprises at least two 40 mg rifaximin SSD IR tablets.

In an embodiment, the invention includes a method of treating sickle cell disease (SCD) in a patient in need thereof comprising administering at least one rifaximin SSD composition to the patient BID fora period of at most 1, 2, 3, 4, 5, 6, 7, 8, 9, 10, 11, or 12 months, wherein the at least one rifaximin SSD composition comprises at least two 40 mg rifaximin SSD IR tablets.

In an embodiment, the invention includes a method of treating sickle cell disease (SCD) in a patient in need thereof comprising administering at least one rifaximin SSD composition to the patient QD fora period of at least 1, 2, 3, 4, 5, 6, 7, 8, 9, 10, 11, or 12 months, wherein the at least one rifaximin SSD composition comprises at least two 40 mg rifaximin SSD IR tablets.

In an embodiment, the invention includes a method of treating sickle cell disease (SCD) in a patient in need thereof comprising administering at least one rifaximin SSD composition to the patient QD for a period of about 1, 2, 3, 4, 5, 6, 7, 8, 9, 10, 11, or 12 months, wherein the at least one rifaximin SSD composition comprises at least two 40 mg rifaximin SSD IR tablets.

In an embodiment, the invention includes a method of treating sickle cell disease (SCD) in a patient in need thereof comprising administering at least one rifaximin SSD composition to the patient QD fora period of at most 1, 2, 3, 4, 5, 6, 7, 8, 9, 10, 11, or 12 months, wherein the at least one rifaximin SSD composition comprises at least two 40 mg rifaximin SSD IR tablets.

In an embodiment, the invention includes a method of preventing or reducing the occurrence or frequency of vaso-occlusive crisis (VOC) in a patient in need thereof comprising administering at least one rifaximin SSD composition to the patient BID for a period of at least 1, 2, 3, 4, 5, 6, 7, 8, 9, 10, 11, or 12 months, wherein the at least one rifaximin SSD composition comprises at least two 40 mg rifaximin SSD IR tablets.

In an embodiment, the invention includes a method of preventing or reducing the occurrence or frequency of vaso-occlusive crisis (VOC) in a patient in need thereof comprising administering at least one rifaximin SSD composition to the patient BID for a period of about 1, 2, 3, 4, 5, 6, 7, 8, 9, 10, 11, or 12 months, wherein the at least one rifaximin SSD composition comprises at least two 40 mg rifaximin SSD IR tablets.

In an embodiment, the invention includes a method of preventing or reducing the occurrence or frequency of vaso-occlusive crisis (VOC) in a patient in need thereof comprising administering at least one rifaximin SSD composition to the patient BID for a period of at most 1, 2, 3, 4, 5, 6, 7, 8, 9, 10, 11, or 12 months, wherein the at least one rifaximin SSD composition comprises at least two 40 mg rifaximin SSD IR tablets.

In an embodiment, the invention includes a method of preventing or reducing the occurrence or frequency of vaso-occlusive crisis (VOC) in a patient in need thereof comprising administering at least one rifaximin SSD composition to the patient QD for a period of at least 1, 2, 3, 4, 5, 6, 7, 8, 9, 10, 11, or 12 months, wherein the at least one rifaximin SSD composition comprises at least two 40 mg rifaximin SSD IR tablets.

In an embodiment, the invention includes a method of preventing or reducing the occurrence or frequency of vaso-occlusive crisis (VOC) in a patient in need thereof comprising administering at least one rifaximin SSD composition to the patient QD for a period of about 1, 2, 3, 4, 5, 6, 7, 8, 9, 10, 11, or 12 months, wherein the at least one rifaximin SSD composition comprises at least two 40 mg rifaximin SSD IR tablets.

In an embodiment, the invention includes a method of preventing or reducing the occurrence or frequency of vaso-occlusive crisis (VOC) in a patient in need thereof comprising administering at least one rifaximin SSD composition to the patient QD for a period of at most 1, 2, 3, 4, 5, 6, 7, 8, 9, 10, 11, or 12 months, wherein the at least one rifaximin SSD composition comprises at least two 40 mg rifaximin SSD IR tablets

The amount of a provided dispersion in the composition will also depend upon the particular compound in the composition. In one aspect, the dosage amount of rifaximin in a provided composition is 40 mg. In another aspect, the dosage amount of rifaximin in a provided composition is 80 mg.

In some embodiments, the soluble solid dispersion tablet will include rifaximin in an amount of about 40 mg and will be an immediate release tablet.

In some embodiments, the amount of rifaximin delivered per dose may be about 40 mg, 80 mg, 120 mg, or 160 mg, which may be provided by delivering, per dose, at least one, two, three, or four 40 mg rifaximin SSD IR tablets, respectively. In some embodiments, such a dose may be delivered QD, BID, TID, or QID.

In some embodiments, the amount of rifaximin delivered per dose may be about 80 mg or 160 mg, which may be provided by delivering, per dose, at least one or two 80 mg rifaximin SSD IR tablets, respectively. In some embodiments, such a dose may be delivered QD, BID, TID, or QID.

In a particular embodiment, the methods of the invention may include delivering 40 mg or 80 mg of rifaximin, per dose, either QD or BID, with 40 mg rifaximin SSD IR tablets (i.e., either 40 mg×1 tablet (40 mg dose) or 40 mg×2 tablets (80 mg dose)).

In some embodiments, the rifaximin compositions described herein may be administered with an additional SCD therapeutic agent in the foregoing methods of treatment. In some embodiments, the additional SCD therapeutic agent may be, for example, hydroxyurea, L-glutamine, hydroxycarbamide, an erythropoietin stimulating agent, and/or an opioid analgesic. In some embodiments, the opioid analgesic may be selected from the group consisting of morphine, codeine, hydrocodone, hydromorphone, methadone, tramadol, oxycodone, tapentadol, fentanyl, and a combination thereof.

### EXAMPLES

### Example 1

### Preparation of Solid Dispersions and Methods of Using the Same

### General Preparation of Solid Dispersions

The solid dispersions described herein can be prepared by a number of methods, including by melting and solvent evaporation. The solid dispersions of the invention described herein can also be prepared according to the procedures described in: Chiou W L, Riegelman S: “Pharmaceutical applications of solid dispersion systems”, J. Pharm. Sci. 1971; 60: 1281-1302; Serajuddin A T M: “Solid dispersion of poorly water-soluble drugs: early promises, subsequent problems, and recent breakthroughs”, J. Pharm. Sci. 1999; 88: 1058-1066; Leuner C, Dressman J: “Improving drug solubility for oral delivery using solid dispersions”, Eur. J. Pharm. Biopharm. 2000; 50:47-60; and Vasconcelos T, Sarmento B, Costa P: “Solid dispersions as strategy to improve oral bioavailability of poor water soluble drugs”, Drug Discovery Today 2007; 12:1068-1075, all of which are incorporated herein by reference in their entireties.

In one aspect, the components, e.g., rifaximin, polymer and methanol are mixed and then spray dried. Exemplary conditions are summarized in Table 1 below.

Exemplary Spray Drying Process Parameters, include for example:
Spray Dryer—e.g., PSD 1;
Single or multi-fluid nozzle: e.g., a two Fluid Niro Nozzle;
Nozzle orifice—0.1-10 mm;
Inlet gas temperature—75-150±5 deg C.;
Process gas flow (mmH2O)—20-70, preferred 44;
Atomizing gas pressure—0.7-1 bar;
Feed rate—2-7 kg/Hr;
Outlet temperature—30-70±3 deg C.;
Solution temperature—20-50 deg C.; and
   Post spray drying vacuum dry at 20-60 deg C., for between about 2 and 72 hrs.

TABLE 1

| | | | | | | Spray rate |
|---|---|---|---|---|---|---|
| Description | Inlet temp. | | | Inlet temp. | Outlet temp. | (b) |
| (a) | (set, ° C.) | Aspirator % | Pump % | (measured, ° C.) | (measured, ° C.) | mL/min |
| (50:50) | 120 | 95 | 40-30 | 120-119 | 60-45 | 9.6 |
| HPMC-AS:rifaximin, ~10 g | | | | | | |
| scale | | | | | | |

(a): approximate ratio of rifaximin to HPMC-AS, by weight.
(b): flow rates are estimated at 30% pump.

A representative batch formula is provided in Table 2.

TABLE 2

| | | | Theoretical |
|---|---|---|---|
| | | | Quantity |
| Ingredient | Function | % w/w | (kg/batch) |
| Rifaximin | Active | 47.20 | 22.18 |
| HPMC-AS | Polymer | 47.20 | 22.18 |
| Poloxamer 407 | Surfactant | 5.60 | 2.64 |
| Methanol^{a} | Solvent | — | (438.0) |
| Nitrogen^{b} | Process gas | — | — |

| | | |
|---|---|---|
| Total Theoretical Weight | 100.00 | 47.00 |

^{a}Removed during drying process;
^{b}process gas for drying and atomization; not incorporated in product

### Blending/Encapsulation Procedure

The components and composition of an 80 mg and 40 mg immediate and sustained extended release tablets are provided in Table 3 below.

TABLE 3

| | | Theoretical Quantity (mg/tablet) | | | |
|---|---|---|---|---|---|
| | | 80 mg- | 80 mg- | 40 | 40 mg- |
| Ingredient | Function | IR | SER | mg-IR | SER |
| Rifaximin | Active | 80 | 80 | 40 | 40 |
| HPMC-AS | Polymer | 80 | 80 | 40 | 40 |
| Poloxamer 407 | Surfactant | 9.49 | 9.49 | 4.75 | 4.75 |
| Croscarmellose sodium | Dissolution | 30.15 | 11.33 | 20.74 | 11.33 |
| | enhancer | | | | |
| Microcrystalline cellulose | Filler | 25.28 | 44.10 | 119.43 | 128.84 |
| Colloidal silicon dioxide | Glidant | 0.45 | 0.45 | 0.45 | 0.45 |
| Magnesium stearate | Lubricant | 1.13 | 1.13 | 1.13 | 1.13 |
| (non-bovine) | | | | | |
| Opadry II Blue 85F90614 | Coating | 11.92 | 11.92 | 11.92 | 11.92 |
| (PVA coating) | | | | | |
| Purified Water | Solvent for | — | — | — | — |
| | coating | | | | |
| | solution | | | | |

| | | | | |
|---|---|---|---|---|
| Total Theoretical Weight | 238.42 | 238.42 | 238.42 | 238.42 |

### Example 2

### Proposed Randomized, Double-Blind, Placebo-Controlled Study to Characterize the Safety, Efficacy, Pharmacokinetics, and Pharmacodynamics of Rifaximin SSD in Sickle Cell Disease Patients with Vaso-Occlusive Crisis

The purpose of this study is to evaluate the safety, efficacy, and pharmacokinetics of rifaximin after oral administration of the SSD formulation in sickle cell disease (SCD) patients with history of vaso-occlusive crisis (VOC). Any potential pharmacokinetic-pharmacodynamic (PK/PD) relationship between rifaximin systemic exposure and potential biomarkers of microbially-associated induction of VOC will also be evaluated. Previous clinical studies have shown rifaximin treatment (i.e., XIFAXAN® 550 mg) to have potential benefit in reducing the number of VOCs and use of intravenous opioid analgesia (IOA). One possible explanation of this benefit may be due to modulation of intestinal microbial composition in SCD patients. Significant elevation of circulating aged neutrophils (CANs), with high CXCR4 and low CD62L surface expression, has been observed during VOCs and has been implicated in the development of the condition. It is proposed that this may occur in response to increased translocation of intestinal bacteria and bacterial products, which may be controlled with the administration of rifaximin.

This study will evaluate the safety, efficacy, and PK of rifaximin in SCD patients, as well as PK/PD relationships between rifaximin and several putative biomarkers associated with the proposed mechanism.

### Objectives

The primary objective of this study is to assess the efficacy of rifaximin SSD in reducing VOCs in SCD patients.

Secondary objectives of this study are: (1) assessment of efficacy of rifaximin SSD in reducing subcategories of VOCs in SCD patients; (2) assessment of rifaximin SSD impact on IOA usage during VOC; (3) assessment of rifaximin SSD impact on outpatient opioid usage; (4) assessment of safety and tolerability of rifaximin SSD in SCD patients; and (5) characterization of PK and the PK/PD relationships between rifaximin SSD and potential biomarkers of microbially-associated VOCs.

### Endpoints

The primary efficacy endpoint of this study will be measured by the annualized rate of VOCs (overall and leading to healthcare visits).

The secondary efficacy endpoints of this study will be measured by the annualized rate of VOCs by subcategory (overall and leading to healthcare visits); the annualized rate of SCD-associated medical facility visits and/or hospitalization visits; and the duration of SCD-associated medical facility visits and/or hospitalization visits.

The secondary endpoint of impact on IOA use for this study is measured by the annualized rate of days using IOA; the time to readiness-for-discharge from first use of IOA during VOC; cumulative IOA consumption during VOC; and time to discontinuation of IOA use during VOC.

The secondary endpoint of assessment of rifaximin SSD impact on outpatient opioid usage is measured in MME units.

The secondary endpoint of safety is measured by AEs, vital signs, and clinical labs.

The secondary endpoint of PK (rifaximin and 25-desacetyl rifaximin in plasma) is measured by subjects with intensive PK sampling (Day 1): Cₘₐₓ, Tₘₐₓ, AUCₗₐₛₜ, AUC₀₋₁₂, AUC_{inf}, λz, t_{1/2}, CL/F, Vz/F, MR__{AUCinf}; subjects with intensive PK sampling (Day 29): C_{trough}, C_{max,ss}, T_{max,ss}, AUCₜₐᵤ, C_{ss,av} λz, t_{1/2}, CL/Fₛₛ, Vz/Fₛₛ, R_{AUC}, R_{Cmax}, MR_AUCₜₐᵤ; and subjects with sparse sampling: (Day 1, Day 8 [±1 day], Day 15 [±1 day], Day 29 [±1 day], Month 3, and Month 6): C_{trough}, Cₘₐₓ, AUC.

The secondary PD endpoint is measured by number and change from predose on Day 1 (at Day 8 [±1 day], Day 15 [±1 day], Day 29 [±1 day], Month 3, and Month 6) for total neutrophils and CANs, serum CD62L, urine 3-indoxyl sulfate, LPS, zonulin, serum citrulline, intestinal fatty-acid binding protein (iFABP).

The secondary PK/PD endpoint will be measured by evaluating the PK/PD relationships between rifaximin PK and each PD endpoint.

Proposed exploratory endpoints include use of a FANLTC questionnaire; examination of relative taxonomic abundance of fecal microbiota at baseline (screening window), Day 29, and Month; examination of iFABP levels; evaluation of CAN levels; evaluation of Zonulin levels; and evaluation of serum LPS levels.

### Patient Population

SCD Patients that have experienced at least 1 VOC in the 12 months prior to enrollment.

### Key Inclusion and Exclusion Criteria

Inclusion Criteria:
Give informed consent.
Has SCD of any genotype (HbSS, HbSC, HbS β-thalassemia).
18 to 70 years of age (inclusive) on day of consent.
Experienced at least 1 VOC within the preceding 12 months prior to Screening. Prior VOC should include occurrence of appropriate symptoms, visit to medical facility and/or healthcare professional, receipt of parenteral opioid or NSAID analgesia or oral opioid.
If receiving hydroxyurea or hydroxycarbamide (HU/HC) or erythropoietin stimulating agents, patient must have been receiving treatment for at least 6 months prior to Screening and plan to maintain the same dose and schedule during the study.
Must meet the following lab values at screening:
   Absolute Neutrophil Count 1.0×10⁹/L
   Platelets 75×10⁹/L
   Hemoglobin (Hgb)≥4.0 g/dL
   Glomerular filtration rate≥45 mL/min/1.73 m² using CKD-EPI formula
   Direct (conjugated) bilirubin≤2.0×ULN
   Alanine transaminase (ALT)≤3.0×ULN
   INR≥2.0
ECOG performance status≤2

Exclusion Criteria:
History of stem cell transplant.
Acute VOC ending within 7 days prior to Day 1 dosing.
Received any blood products within 30 days of Day 1 dosing.
Uncontrolled liver disease or renal insufficiency, colitis, or inflammatory bowel disease.
Received active treatment on another investigational trial or has taken penicillin prophylaxis or antibiotics for treatment of infection within 30 days or 5 half-lives of the treatment, whichever is greater, prior to screening.
Significant medical condition that requires hospitalization (other than SCD with VOC) within 2 months prior to screening.
Participating in a chronic transfusion program (pre-planned series of transfusions for prophylactic purposes).
Planning on undergoing an exchange transfusion during the duration of the study; episodic transfusion in response to worsened anemia or VOC is permitted.
Hypersensitivity to rifaximin, rifampin, rifamycin antimicrobial agents, or any components of rifaximin SSD.
Use of therapeutic anticoagulation (prophylactic doses permitted) or antiplatelet therapy (other than aspirin or NSAIDs) within the 10 days prior to Day 1 dosing.
Pregnant or nursing women.
Women of child-bearing potential, defined as all women physiologically capable of becoming pregnant, unless tested negative by serum pregnancy test at screening and agrees to standard prevention methods.
History of drug abuse, documented or in opinion of investigator.
Requirement for use of any medications on the prohibited medications list (CYP3A4 inhibitors/inducers, PPIs, PgP substrates).
Any prior gastrointestinal surgery which has altered the anatomy of the esophagus, stomach, or small/large intestine (exceptions include appendectomy, cholecystectomy, and fundoplication).
Colonoscopy or sigmoidoscopy, or any other use of bowel prep, laxative, or enema, within 30 days prior to Day 1 or plans to undergo such a procedure during the duration of the study.
Any documented history of clinical stroke or intracranial hemorrhage, or an uninvestigated neurologic finding within the 12 months prior to screening. Silent infarct only present on imaging is allowed.
Patients with bleeding disorders.
Planning to undergo a major surgical procedure during the duration of the study
Positive for HIV or other concomitant immunodeficiency.
Active Hepatitis B infection (HBsAg positive). Prior infection but not active (i.e., anti-HBc positive, HBsAg and HBV-DNA negative) is allowed.
Positive for Hepatitis C (HCV RNA). Prior infection with spontaneous resolution or sustained resolution after antiviral treatment (i.e., no detectible HCV RNA) for 6 months (with IFN-free treatments) or for 12 months (with use of IFN treatment) after cessation of antivirals are allowed.
Malignant disease. Exceptions include malignancies that were treated curatively and have not recurred within 2 years prior to study treatment, completely resected basal cell and squamous cell skin cancers, and any completely resected carcinoma in situ.
Serious mental or physical illness which, in the opinion of the Investigator, would compromise participation in the study.
Any condition which, in the opinion of the Investigator, is likely to interfere with the successful collection of the measurements required for the study.
Resting QTcF≥470 msec at screening.
Cardiac or cardiac repolarization abnormality, including any of the following:
   History of myocardial infarction (MI) angina pectoris, coronary artery bypass graft (CABG), or uncontrolled congestive heart failure within 6 months prior to Day 1.
   Clinically significant cardiac arrhythmias (e.g., ventricular tachycardia), complete left bundle branch block, high-grade AV block (e.g., bifascicular block, Mobitz type II and third-degree AV block).
   Long QT syndrome, family history of idiopathic sudden death or congenital long QT syndrome, or any of the following:
      Risk factors for Torsade de Pointes (TdP) including uncorrected hypokalemia or hypomagnesemia, history of cardiac failure, or history of clinically significant/symptomatic bradycardia.
      Concomitant medications with a known risk of TdP that cannot be discontinued or replaced by safe alternative (within 5 half-lives prior to starting study drug).
      Inability to determine the QTcF interval.
Not able to understand or comply with study instructions and requirements.
For subjects in intensive PK group, subjects with hepatic impairment (Child-Pugh Class A, B, or C) should be excluded.

### Dosing Regimen

Rifaximin SSD 40 mg IR tablets are intended to be used for this study as the investigational drug with a placebo reference. The proposed design is a randomized 1:1:1 to placebo, 40 mg rifaximin SSD IR treatment, and 80 mg rifaximin SSD IR treatment (40 mg×2). Scheduled visits at Day 1, Day 8 (+/−1 day), Day 15 (+/−1 day), Day 29 (+/−1 day), Month 3, and Month 6.

The dosing duration and frequency will be an assigned treatment taken orally twice daily (BID) for 6 months. The cohorts for this study are (1) placebo, (2) 40 mg rifaximin SSD IR (40 mg×1), and (3) 80 mg rifaximin SSD IR (40 mg×2).

### Sample Size

The sample size will be 150 subjects (50 per treatment group) with at least 10 subjects providing intensive PK in each group.

A sample size of at least 50 subjects per group will provide at least 90% power, at an alpha level of 0.05, to detect a 55% lower rate of VOC with rifaximin 80 mg than with placebo, using Wilcoxon rank-sum test. A median rate of VOC in the placebo group of 2.25 is assumed during the study duration, with standard deviation of 1.7.

A hierarchical testing procedure will be used (alpha level of 0.05 for high-dose rifaximin vs. placebo and, if significant, low-dose rifaximin vs. placebo). Nominal p-values will be presented for all comparisons.

### Subject Assessments

Efficacy assessments to be made include:
Number of VOCs during treatment and history thereof for 12 months prior to treatment. Crises identified by trial investigators will be adjudicated in a blinded fashion by an independent crisis-review committee.
Number of VOCs by subcategory (uncomplicated pain crisis, acute chest syndrome, hepatic sequestration, splenic sequestration, priapism) during treatment and history thereof for 12 months prior to treatment. Crises identified by trial investigators will be adjudicated in a blinded fashion by an independent crisis-review committee.
Number of SCD-associated hospitalization and ER visits during treatment and history thereof for 12 months prior to treatment.
Date and time for each start and stop of IOA use during VOCs.
Duration of hospitalization during each VOC, date/time of first use of IOA, and date/time of readiness-for-discharge.
Cumulative consumption of IOAs during each VOC during treatment and history thereof for 12 months prior to treatment.
Cumulative time of IOA usage during each VOC.

Safety and tolerability assessments include AEs, vital signs, clinical labs, and ECGs.

Other assessments include:
Functional Analysis of Non-life-Threatening Conditions (FANLTC) questionnaire predose on Day 1, at Day 29, 3 months, and 6 months.
Stool sample for microbiome profiling during screening window, at Day 29 and at 6 Months.

PK assessments will be provided as follows:
For intensive PK Subjects (Day 1 & Day 29): Predose, 0.5, 1, 1.5, 2, 3, 4, 5, 8, and 12 hr post dose (12 hr timepoint should be prior to second dose). For all other scheduled visits, predose and up to 3 additional post-dose samples (TBD by pharmacometrician).
For subjects with Sparse Sampling: Predose and up to 3 additional post-dose samples (TBD by pharmacometrician) on Day 1, Day 8, Day 15, Day 29, Month 3, and Month 6.
For all subjects: During medical facility visit for VOC with estimated time of last dose, when possible.
Dosing diary kept with time of dose recorded.

PD assessments will be provided as follows:
Predose on Day 1, during Day 8, Day 15, Day 29, Month 3, and Month 6 visits, and during medical facility visit for VOC when possible:
   Neutrophil markers: Total Neutrophils (Count and % WBC), CANs (Count and % Neutrophils), Serum CD62L.
Gut permeability markers: Zonulin, serum citrulline, iFABP.
   Gut bacteria markers: LPS, Urine 3-indoxyl sulfate.

### Data Analysis

The primary efficacy end point is the annual rate of VOC, which will be calculated as follows: total number of adjudicated VOC×365÷(end date−date of randomization+1), with the end date defined as the date of the last dose plus 14 days. The difference in the annual VOC rate for each rifaximin group versus the placebo group will be analyzed with a Wilcoxon rank-sum test, stratified by use of categorized history of crises in the previous year (<5; >=5 POV).

Change from Baseline in rate of VOCs, days using IOA, SCD-associated hospitalization and ER events and duration will be summarized by treatment and compared to placebo.

Time to readiness-for-discharge from first use of IOAs during each VOC and time to discontinuation of IOA use during VOCs will be summarized by treatment group with descriptive statistics and presented as Kaplan-Meier plots.

Cumulative use of IOA consumption during VOCs will be summarized by treatment group with descriptive statistics and compared to placebo.

AEs to be summarized by MedDRA System Organ Class (SOC) and Preferred Term (PT) and reported by treatment group and relationship to treatment. Observed and Change from Baseline in Vital Signs, labs, and ECG parameters (RR, PR, QTcF, QRS)

PK will be evaluated in intensive PK subjects using noncompartmental analysis. A population PK model will be developed using data from all subjects providing quantifiable post-dose samples. Steady-state will be assessed for all subjects using Cirough measurements on Day 8, Day 15, and Day 29, and may be simulated using the population PK model.

PD endpoints will be summarized by treatment with quantity and change from baseline at each visit.

Relationship between rifaximin PK parameters and each PD endpoint will be evaluated using ANOVA models. A population PK/PD model may be developed as a separate analysis if warranted.

Change from Baseline in FANLTC questionnaire scores will be summarized by treatment and compared to placebo.

Change from Baseline in intestinal microbiome composition may be summarized by treatment and compared to placebo

### Conclusion

Following performance of the foregoing study, it is expected that a 40 mg rifaximin SSD IR formulation (either as 40 mg or 80 mg (40 mg×2 tablets)) will yield the surprising benefit of reducing VOCs in SCD patients in an oral dosage formulation that contains substantially less rifaximin than that provided in Xifaxan® 550 mg.

Based on such performance, it is further expected that delivery of the rifaximin SSD IR formulation described herein will result in a reduction in IOA usage by subjects during VOC.

Accordingly, it is expected that the rifaximin SSD IR formulation described herein (e.g., rifaximin SSD 40 mg IR) will demonstrate surprising efficacy for the treatment of SCD in patients as will be demonstrated by the foregoing study.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

### Further Embodiments

Embodiment 1. A rifaximin SSD composition for use in a method of treating sickle cell disease (SCD) in a patient in need thereof.

According to a preferred embodiment at least one rifaximin SSD composition is administered to the patient.

Embodiment 2. A rifaximin SSD composition for use in a method of reducing elevated levels of circulating aged neutrophils (CANs) in a patient in need thereof.

According to a preferred embodiment at least one rifaximin SSD composition is administered to the patient.

Embodiment 3. A rifaximin SSD composition for use in a method of treating vaso-occlusive crises (VOCs) in a patient in need thereof.

According to a preferred embodiment at least one rifaximin SSD composition is administered to the patient.

Embodiment 4. The composition for use according to any of the preceding embodiments, wherein the at least one rifaximin SSD composition comprises an immediate release (IR) tablet, a sustained extended release (SER) tablet, or a combination thereof.

Embodiment 5. The composition for use according to any of the preceding embodiments, wherein the at least one rifaximin SSD composition comprises at least one 40 mg rifaximin SSD IR tablet, an 80 mg rifaximin SSD IR tablet, a 40 mg SSD SER tablet, an 80 mg SSD SER tablet, or a combination thereof.

Embodiment 6. The composition for use according to any of the preceding embodiments 1-4, wherein the at least one rifaximin SSD composition comprises at least one rifaximin SSD IR tablet.

Embodiment 7. The composition for use according to embodiment 6, wherein the at least one rifaximin SSD IR tablet comprises a 40 mg rifaximin SSD IR tablet, an 80 mg rifaximin SSD IR tablet, or a combination thereof.

Embodiment 8. The composition for use according to any of the preceding embodiments, wherein the at least one rifaximin SSD composition is administered to the patient QD, BID, TID, or QID.

Embodiment 9. The composition for use according to embodiment 8, wherein the at least one rifaximin SSD composition is administered to the patient QD or BID.

Embodiment 10. The composition for use according to embodiment 9, wherein the at least one rifaximin SSD composition is administered to the patient BID.

Embodiment 11. The composition for use according to any of the preceding embodiments, wherein the at least one rifaximin SSD composition is administered to the patient for a period of at least 1, 2, 3, 4, 5, 6, 7, 8, 9, 10, 11, or 12 months.

Embodiment 12. The composition for use according to any of the embodiments 1-10, wherein the at least one rifaximin SSD composition is administered to the patient for a period of at most 1, 2, 3, 4, 5, 6, 7, 8, 9, 10, 11, or 12 months.

Embodiment 13. The composition for use according to any of the embodiments 1-10, wherein the at least one rifaximin SSD composition is administered to the patient for a period of about 1, 2, 3, 4, 5, 6, 7, 8, 9, 10, 11, or 12 months.

Embodiment 14. The composition for use according to any of the preceding embodiments, wherein the at least one rifaximin SSD composition comprises at least two, three, or four rifaximin SSD tablets.

Embodiment 15. The composition for use according to embodiment 14, wherein the at least one rifaximin SSD composition comprises at least one, two, three, or four 40 mg rifaximin SSD tablets.

Embodiment 16. The composition for use according to embodiment 14, wherein the at least one rifaximin SSD composition comprises at least one, two, three, or four 40 mg rifaximin SSD IR tablets.

Embodiment 17. The composition for use according to embodiment 14, wherein the at least one rifaximin SSD composition comprises at least one or two 80 mg rifaximin SSD tablets.

Embodiment 18. The composition for use according to embodiment 14, wherein the at least one rifaximin SSD composition comprises at least one or two 80 mg rifaximin SSD IR tablets.

Embodiment 19. The composition for use according to any one of the preceding embodiments, wherein furthermore an additional SCD therapeutic agent is administered to the patient.

Embodiment 20. The composition for use according to embodiment 19, wherein the additional SCD therapeutic agent comprises hydroxyurea, L-glutamine, hydroxycarbamide, an erythropoietin stimulating agent, an opioid analgesic, or a combination thereof.

Embodiment 21. The composition for use according to embodiment 20, wherein the opioid analgesic comprises morphine, codeine, hydrocodone, hydromorphone, methadone, tramadol, oxycodone, tapentadol, fentanyl, or a combination thereof.

Embodiment 22. The composition for use according to any one of embodiments 3 to 21, wherein the method of treating vaso-occlusive crises (VOCs) in the patient in need thereof comprises alleviating one or more symptoms of VOCs in the patient.

Embodiment 23. The composition for use according to any one of embodiments 3 to 21, wherein the method of treating vaso-occlusive crises (VOCs) in the patient in need thereof comprises reducing or preventing the occurrence of VOCs in the patient.

Embodiment 24. The composition for use according to any one of embodiments 3 to 21, wherein the method of treating vaso-occlusive crises (VOCs) in the patient in need thereof comprises reducing the duration or severity of VOCs in the patient.

Embodiment 25. The composition for use according to any one of embodiments 3 to 21, wherein the method of treating vaso-occlusive crises (VOCs) in the patient in need thereof comprises mediating or otherwise reducing the patient's opioid usage during VOCs.

1. A method of treating sickle cell disease (SCD) in a patient in need thereof comprising administering at least one rifaximin SSD composition to the patient.

2. A method of reducing elevated levels of circulating aged neutrophils (CANs) in a patient in need thereof comprising administering at least one rifaximin SSD composition to the patient.

3. A method of treating vaso-occlusive crises (VOCs) in a patient in need thereof comprising administering at least one rifaximin SSD composition to the patient.

4. The method according to claim 1, wherein the at least one rifaximin SSD composition comprises an immediate release (IR) tablet, a sustained extended release (SER) tablet, or a combination thereof.

5. The method according to claim 1, wherein the at least one rifaximin SSD composition comprises at least one 40 mg rifaximin SSD IR tablet, an 80 mg rifaximin SSD IR tablet, a 40 mg SSD SER tablet, an 80 mg SSD SER tablet, or a combination thereof.

6. The method according to claim 1, wherein the at least one rifaximin SSD composition comprises at least one rifaximin SSD IR tablet.

7. The method according to claim 6, wherein the at least one rifaximin SSD IR tablet comprises a 40 mg rifaximin SSD IR tablet, an 80 mg rifaximin SSD IR tablet, or a combination thereof.

8. The method according to claim 1, wherein administering the at least one rifaximin SSD composition to the patient comprises delivering the at least one rifaximin SSD composition QD, BID, TID, or QID to the patient.

9. The method of claim 8, wherein administering the at least one rifaximin SSD composition to the patient comprises delivering the at least one rifaximin SSD composition QD or BID to the patient.

10. The method of claim 9, wherein administering the at least one rifaximin SSD composition to the patient comprises delivering the at least one rifaximin SSD composition BID to the patient.

11. The method according to claim 1, wherein administering the at least one rifaximin SSD composition to the patient comprises delivering the at least one rifaximin SSD composition to the patient for a period of at least 1, 2, 3, 4, 5, 6, 7, 8, 9, 10, 11, or 12 months.

12. (canceled)

13. (canceled)

14. The method according to claim 1, wherein the at least one rifaximin SSD composition comprises at least two, three, or four rifaximin SSD tablets.

15. (canceled)

16. The method of claim 14, wherein the at least one rifaximin SSD composition comprises at least one, two, three, or four 40 mg rifaximin SSD IR tablets.

17. (canceled)

18. The method of claim 14, wherein the at least one rifaximin SSD composition comprises at least one or two 80 mg rifaximin SSD IR tablets.

19. The method according to claim 1, further comprising administering an additional SCD therapeutic agent to the patient.

20. The method of claim 19, wherein the additional SCD therapeutic agent comprises hydroxyurea, L-glutamine, hydroxycarbamide, an erythropoietin stimulating agent, an opioid analgesic, or a combination thereof.

21. (canceled)

22. The method according to claim 3, wherein the method of treating vaso-occlusive crises (VOCs) in the patient in need thereof comprises alleviating one or more symptoms of VOCs in the patient.

23. The method according to claim 3, wherein the method of treating vaso-occlusive crises (VOCs) in the patient in need thereof comprises reducing or preventing the occurrence of VOCs in the patient.

24. The method according to claim 3, wherein the method of treating vaso-occlusive crises (VOCs) in the patient in need thereof comprises reducing the duration or severity of VOCs in the patient.

25. The method according to claim 3, wherein the method of treating vaso-occlusive crises (VOCs) in the patient in need thereof comprises mediating or otherwise reducing the patient's opioid usage during VOCs.
